(19) United States
(12) Patent Application Publication
(10) Pub. No.: US 2018/0155464 A1
(43) Pub. Date: Jun. 7, 2018

(54) DIALKYLCARBOXYLATE-AROMATIC-FUNCTIONALIZED POLYMERS THAT DO NOT RELEASE ENDOCRINE DISRUPTING COMPOUNDS

(71) Applicant: The Regents of the University of California, Oakland, CA (US)

(72) Inventor: Rebecca Braslau, Santa Cruz, CA (US)

(73) Assignee: The Regents of the University of California, Oakland, CA (US)

(21) Appl. No.: 15/823,551

(22) Filed: Nov. 27, 2017

Related U.S. Application Data
(62) Division of application No. 14/440,840, filed on May 5, 2015, now abandoned.
(60) Provisional application No. 61/880,964, filed on Sep. 22, 2013.
(60) Provisional application No. 61/729,717, filed on Nov. 26, 2012.
(60) Provisional application No. 61/722,346, filed on Nov. 5, 2012.

(51) Int. Cl. C08F 8/30; C07D 249/04; C08L 27/22; C08L 25/18

(52) U.S. Cl. CPC C08F 8/30; C07D 249/04; C08L 25/18; C08L 27/22

(57) ABSTRACT

Disclosed are novel phthalate compounds and a simple and economical route to covalently attach a phthalate ester mimic to PVC is described, allowing plasticization of PVC without the danger of Endocrine Disruption Chemicals leaching from the polymer matrix. An azide-alkyne Huisgen cycloaddition (in the absences of copper catalyst) using dialkyl acetylenedicarboxylates allows this cycloaddition to occur under very mild thermal conditions.

### RELATED APPLICATIONS

This application is a divisional of U.S. application Ser. No. 14/440,840, filed May 5, 2015, which is a National Stage of International Application No. PCT/US2013/068410, filed Nov. 5, 2013, which claims the benefit of U.S. Provisional Application No. 61/722,346, filed Nov. 5, 2012, U.S. Provisional Application No. 61/729,717, filed Nov. 26, 2012, and U.S. Provisional Application No. 61/880,964, filed Sep. 22, 2013. The only inventor on all these applications is Dr. Rebecca Braslau. All of these applications, U.S. Patent Application Publication No. 2015-0299343, published Oct. 22, 2015, International Application Publication No. WO/2014/071347, published May 8, 2015, and U.S. Provisional Application No. 61/594,052, filed Feb. 2, 2012 (and all cited literature and publications herein) are hereby incorporated by reference in their entirety for all purposes.

### FIELD OF THE INVENTION

Plasticizing agents used for changing the physical qualities of commercial polymers.

### BACKGROUND

Plasticizers are compounds added to a material to decrease brittleness and increase the plasticity or fluidity of the material. The most common applications are for plastics, especially polyvinyl chloride (PVC). Traditional Plasticizers work by embedding themselves between chains of polymers, with no covalent bonds being formed, thereby spacing the polymer chains apart and increasing the “free volume”, thus lowering the glass transition temperature for the plastic and making it softer.

Phthalates (also called phthalate esters) are esters of phthalic acid (1,2-benzenedicarboxylic acid) and are mainly used as plasticizers. Plasticizers are compounds that are added to plastics to alter their flexibility, transparency, durability, stiffness and longevity, frequently increasing plastic qualities such as malleability and decreasing brittleness. They are primarily used to soften polyvinyl chloride (PVC) with almost 90% of the market for plasticizers being used for PVC, providing improved flexibility and durability.

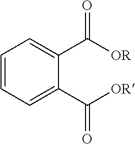

Shown above is a generic chemical structure of a phthalate. R and R′═CₙH₂ₙ₊₁; n=4-15.

Since the 1930's small molecule phthalate esters have been used very commonly (approx. 6 million tons per year) for the formulation of PVC consumer products. Phthalates are relativly easily leached from the plastic matrix into the environment due to the fact that there is no covalent bond between the phthalates and plastics in which they are mixed. As plastics age and break down, the rate of release of phthalates accelerates.

In use, phthalate esters leach from the polymer matrix, and when metabolized, can give rise to molecules that can bind to and act upon endocrine receptors for mammals, reptiles, amphibians and bird. This is because the leached phthalate esters can structurally and functionally resemble hormones, and therefore act as endocrine disruptors.

These endocrine disruptors are implicated in a variety of serious health problems including male and female reproductive tract abnormalities, and feminization, miscarriage, menstrual problems, changes in hormone levels, early puberty, brain and behavior problems, impaired immune functions, developmental abnormalities, infertility and cancer. These dangers have been recognized and phthalate plasticizers have been banned from a number of specific applications including child care products and some toys. The use of the specific phthalate esters DEHP, DBP (dibutyl phthalate) and BBP (butylbenzyl phthalate) in toys and other child care articles was forbidden by the European Union in 2005, and was banned by the Consumer Safety Commission in 2009 in the United States for toys marketed to children younger than 12 years old, and child care articles for children up to age 3. But phthalate plasticizers continue to be used for food packaging, medical devices and some toys, and also in articles such as rain coats and cosmetics. Clearly there is a need for alternative plasticizers that do not pose such risks.

### BRIEF SUMMARY OF THE INVENTION

The invention encompasses a novel, simple and economical method of covalently attaching a phthalate ester mimic to polymers such as PVC, allowing plasticization of PVC and other polymers to produce commercial polymers from which endocrine disruption chemicals do not leach (or leach in very small quantities) from the polymer matrix. The invention also encompasses the products of such methods, as well as methods for making and using such compounds and plastics (such as PVC) blended with such compounds.

The invention (supported by PCT/US13/24582 and U.S. Application No. 61/594,052, filed 2 Feb. 2012, both of which are incorporated by reference in this and the prior international application) additionally encompasses polyphthalate compounds comprising polymers containing pendant phthalate esters that under common environmental conditions do not release phthalate esters (which are known to be endocrine disruptors) in any significant amount. The invention also encompasses methods for making such compounds. The methods of making the compounds of the invention may use any suitable type of polymerization reaction, many of which are known in the art. For example, Nitroxide Mediated Radical Polymerization (NMRP) may be used. Other controlled and uncontrolled polymerization reactions may be used with one or more type of monomer reactant. The invention also encompasses compositions comprising a plastic in need of plasticization (such as PVC) blended with such compounds, and articles made with such compositions.

In an exemplary embodiment, polymers containing pendant phthalate esters are prepared by Nitroxide Mediated Radical Polymerization (NMRP). The products may be used as plasticizers when blended with polyvinyl chloride (PVC) and other plastics.

In another exemplary embodiment of this new process, 4-vinylphthalate ester monomers are polymerized in a controlled manner by NMRP to give short polymers consisting of a covalent carbon chain backbone bearing phthalate ester side-groups. Hydrolysis of these designed polymers will release only alcohols, rather than phthalates. Thus degradation products cannot be metabolized to give hormone mimics that may cause endocrine disruption. Both homo- and copolymers are prepared by the method of the invention, allowing the preparation of polymers with variable molecular weights, variable spacing between phthalate moieties, and variable polarity.

In one disclosed exemplary embodiment, the applicants used a 4-vinylphthalate ester, however, the invention equally encompasses use of vinylphthalate esters with different substitutions, for example, 3, 4, 5 or 6 vinylphthalate esters (positions 1 and 2 already being occupied by the ester groups). All such variations are encompassed in the invention and all such variations should provide the same functional benefits.

The invention encompasses (but is not limited to) the following embodiments:

A polyphthalate polymer compound comprising low molecular weight (2000-25000) short (Degree of Polymerization=9-130) polystyrene or polyacrylate or polyacrylamide polymers bearing pendant phthalate esters, that under experimental conditions described below release no detectable amount (less than 1% or alternatively less than 3%) of phthalate esters. These experimental conditions are as follows: Hydrolysis of PVC/polyphthalate films are performed by aging the films for 10 weeks at 37° C., and alternatively at 70° C. in water at neutral and low pH following the procedure described in: Wang, Q.; Storm, B. K., Migration of additives from poly(vinyl chloride) (PVC) tubes into aqueous media. Macromolecular Symposia 2005, 225, 191-203.

A polyphthalate compound comprising polymers containing pendant phthalate esters that under experimental conditions release no detectable amount (less than 1% or alternatively less than 3%) of phthalate esters. An assay used to measure the release of phthalate esters by hydrolysis of PVC/polyphthalate films may be performed by aging the films for 10 weeks at 37° C. (alternatively at 50° C., 60° C. or 70° C.) in water at neutral (and alternatively low) pH following the procedure described by Wang (see above).

A method for altering the physical properties of PVC by mixing the PVC with a plasticizer, wherein the plasticizer is a polyphthalate polymer compound comprising low molecular weight (2000-25000) polymers having pendant phthalate esters that under experimental conditions do not release phthalate esters. In alternative embodiments, the molecular weight of the polymers may be, for example, 500 to 2500000, or 1000 to 2000000, or 1500 to 1000000, or 2000 to 500000. Mixing may be done, for example, by solution casting (Lindstrom, et al., Journal of Applied Polymer Science 2006, 100, (3), 2180-2188) or may be done by any other suitable method of mixing such as simple mechanical mixing at a temperature that encourages blending of components. No covalent bonds are formed between the PVC and plasticizer. The plasticizer is a polyphthalate polymer compound comprising a preferably low molecular weight (2000-25000) polymer (Degree of Polymerization=9-130), for example a polystyrene or polyacrylate or polyacrylamide polymer (or mixtures thereof) bearing pendant phthalate esters, that under standard experimental conditions (described herein) release no measurable phthalate esters (or in other embodiments, release <1%, or <3%, or <10%, or <20%, or <30% phthalate esters over a fixed and defined time, such as 10 weeks).

A method for preparing a polyphthalate compound, the compound comprising low molecular weight (2000-25000) short (Degree of Polymerization=9-130) polystyrene or polyacrylate or polyacrylamide polymers bearing pendant phthalate esters, that under experimental conditions described herein release no detectable amount (less than 1% or alternatively less than 3%) of phthalate esters; the method comprising polymerizing 4-vinylphthalate ester monomers in a controlled manner using Nitroxide Mediated Radical Polymerization (NMRP) wherein polymerization is carried out at a temperature between, for example 120° C. and 126° C. using unimolecular alkoxyamine initiators. Alternatively a higher or lower temperature range may be used, for example, temperatures may be 110° C. to 140° C. or 40° C. to 200° C. or 60° C. to 175° C. or 80° C. to 155° C. or 100° C. to 150° C. or 100° C. to 135° C. or 110° C. and 130° C. The polymerization can be carried out neat (without a solvent) in the monomer without the presence of a solvent to produce short polymers of a covalent carbon backbone bearing phthalate ester side-groups. Alternatively a solvent may be used for the polymerization reaction. Alternatively polymerization may be done by any other known method.

Preparation of Poly(Phthalate) Plasticizers

The present invention entails preparation of poly(vinylphthalate ester)s, as homopolymers, or random copolymers with styrene, acrylates or other comonomers, to be used as substitutes for standard phthalate plasticizers in PVC. As phthalate esters are now used on the million ton scale annually, a substitute is desirable that is chemically similar, but which shows low migratory ability from the PVC matrix. As opposed to the current polyester plasticizers, these poly(vinylphthalates) are linked together by a robust carbon polymer backbone: hydrolysis will release only alcohols, rather than phthalates. Thus degradation products pose no danger of being metabolized to form Endocrine Disruptor Compounds.

Any appropriate method of polymerization may be used, and many are known in the art and commercial kits are available. In the present exemplary case, Nitroxide-mediated radical polymerization (NMRP) was used to prepare short homopolymers or copolymers with low polydispersities and predictable molecular weights. FIG. 1 illustrates a general scheme for the development of polymeric phthalate esters by NMRP (as plasticizers). The inventors developed a number of nitroxides as mediators in this process. For vinylphthalate homopolymers the simple TEMPO-based alkoxyamine initiator based on TEMPO 12 (both available commercially from Sigma Aldrich Co.) is effective at producing well-defined polymers. For poly(vinylphthalate-co-acrylate)s, the alkoxyamine based on the alpha-hydrogen bearing nitroxide TIPNO (T-butyl-isopropyl-phenyl-nitroxide), which was developed in the inventor's (Dr. Braslau's) lab, was used to form controlled polymers of predictable composition. Alternatively, and preferably for commercial applications, the very similar initiator BLOCKBUILDER® initiator based on the alpha-hydrogen bearing nitroxide SG1 (13) is available commercially on large scale from Arkema Inc.

FIG. 2 shows Nitroxides and their corresponding N-alkoxyamine initiators for preparing polymeric phthalates.

NMP is an attractive method for this application. The polymerizations are typically carried out by heating in the range of 120-126° C. (other temperature ranges are possible—see above) using unimolecular alkoxyamine initiators, neat in the monomer (alternatively a solvent may be used). The radical nature of the polymerization makes this a chemoselective technique that is tolerant to a variety of functional groups on the monomers including esters, anhydrides, amides, alcohols, amines, epoxides, nitriles, and carbamates. The “Living” nature of the radical polymerization gives polymers of predicable molecular weights (e.g., PDI from about 1.2 to 1.8), and as no solvent is employed, the polymerizations are economical and scaleable to prepare bulk commodities at the industrial level. In some embodiments, solvent may be employed (but generally is not). Solvent may be added to ensure solubility of all of the components. In some embodiments super critical CO2 may be used as the solvent.

### Monomer Synthesis

Although the following method was employed in the current work, it is well known in the art that there are many effective ways to couple the vinyl group onto the aryl bromide (or aryl iodide or aryl tosylate, etc). For the purpose of this investigation, the synthesis of 4-vinylphthalic esters is initially carried out by the most convenient manner possible, without regard to possible palladium catalyst impurities or economic concerns about scale-up. Once effective poly(phthalic ester) plasticizers are developed, routes that are economical and do not entail the use of potentially toxic catalysts become a priority.

The only literature method for the preparation of 4-vinylphthalic acid 15 is that of Stadler, utilizing a Heck reaction in an autoclave with 40 bars of ethylene gas (illustrated in FIG. 3). This group made copolymers of 4-vinylphthalic acid, 4-vinylphthalic anhydride, and 4-vinylphthalic esters with styrene using uncontrolled AIBN-initiated radical polymerization, with the aim of making heat resistant polystyrenes and polymer blends with enhanced impact strength.

The inventors have alternatively used a different synthesis method as described in the following reference, hereby incorporated by reference: JR. Braslau, et al “Polymeric phthalates: potential non-migratory macromolecular plasticizers,” Journal of Polymer Science Part A: Polymer Chemistry, 2013, 51, 1775-1184. (Mar. 1, 2013): doi: 10.1002/pola.26485.

As the 4-vinylphthalates are expected to be prone to polymerization upon handling and storage, it is prudent to prepare esters from the acid prior to introducing the vinyl group. Rather than carry out a Heck reaction it is known in the art that other methods may be used to couple the vinyl group onto the aryl bromide (or aryl iodide or aryl tosylate, etc).

A number of vinyl boron agents have been used to carry out this transformation on aryl bromides, including protocols by Molander and Najera with potassium vinyltrifluoroborate. Joucla has developed a heterogeneous palladium catalyst to be used with potassium vinyltriflouroborates. Alternatively, the use of vinyl boronic acids have been used by a number of research groups. Burke has developed air-stable MIDA boronates to replace unstable vinyl boronic acids in this Suzuki-Miyaura coupling. FIG. 4 illustrates a synthesis of 4-vinylphthalic esters 17 via Szuki-Miyaura coupling.

A much older approach entails preparation of an alkynyl phthalate ester 19 via a Sonogashira coupling, followed by alkyne deprotection upon treatment with base with loss of acetone to give alkynylphthalic esters 20. The chemoselective reduction of the triple bond to the vinyl group should be straightforward.

Denmark has developed the use of very inexpensive and stable vinylpolysiloxane 18 with tetra-butylammonium fluoride as an activator in a related palladium catalyzed coupling (Denmark's Pd cat. vinylsilane coupling with aryl bromides is illustrated in FIG. 5). This vinyl silane/TBAF system employs 5% PdBr2, 5% 2-(di-tertbutylphosphino) biphenyl ligand. Electron poor aryl bromides react quickly compared to electron rich substrates, making this a very attractive method. Yamakawa has also developed a nickel catalyzed coupling of aryl bromides with vinyl ZnBr.MgBrCl.

FIG. 6 shows Sonogashira route to vinyl phthalates.

A less exotic approach is the Diels-Alder reaction between acetylene dicarboxylate esters 21 and the diene 22 developed by Tsuge. Esterification of acetylene dicarboxylic acid to the diester 22 is carried out by standard acid catalyzed Fischer esterification conditions. Treatment of the Diels-Alder product with DBU resulted in elimination reactions to give a mixture of the desired product 25 as the minor component, and the benzyl ether 24 as the major product. It should be possible to convert this mixture to the desired vinyl phthalate ester by treatment with acid.

FIG. 7 shows Diels-Alder approach to 4-vinylphthalates based on the work of Tsuge.

Use of 4-vinylphthalic anhydride 26 as a monomer will allow post-polymerization modification of the polymer, but will result in incomplete esterification, leaving behind some carboxylic acid residues. However, incorporation of some phthalic anhydride into the polymer may prove to be a useful functional handle in allowing incorporation of other functional groups (such as imide formation) subsequent to polymerization. The group of Stadler prepared 4-vinylphthalic anhydride 26 from 4-vinylphthalic acid 15 by sublimation (illustrated in FIG. 8). In an analogous fashion, 4-bromophthalic acid should be easily converted in to the corresponding anhydride, followed by vinylation. This will allow for easier handling of the intermediates, without concerns of premature polymerization during storage.

Various alcohols may be used to prepare the vinylphthalate esters. The most common plasticizer is DEHP, thus 2-ethylhexyl alcohol is an obvious first choice. The alcohols shown in Table 1 is investigated. The differences in branching, molecular weight, and polarity are all expected to affect the free volume created in the PVC polymer, thus contributing to the efficacy of the polymeric plasticizers.

TABLE 1
Alcohols in phathalate plasticizers

| | phthalate |
|---|---|
| | being |
| Alcohols | mimicked |
| 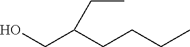 | DEHP |
| 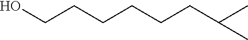 | DINP |
| 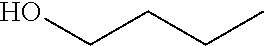 | DBP |
| 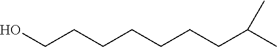 | DIDP |
| 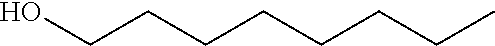 | DOP |
| 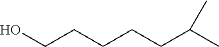 | DIOP |
| 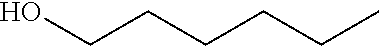 | DNHP |
| 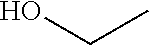 | DEP |
| 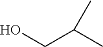 | DIBP |
| 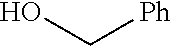 | (half of) BBzP |

Polymerization by Nitroxide-Mediated Radical Polymerization (NMRP)

With the 4-vinylphthalate ester monomers in hand, polymerizations to form homopolymers and copolymers is carried out using NMRP. The resulting polymers is initially characterized by ¹H-NMR of the crude polymerization mixture to determine the ratio of consumed to residual monomer, and then GPC to determine molecular weight and polydispersity.

Homopolymers Short homopolymers using 4-vinylphthalate esters are prepared, varying the molecular weights between 2,000-25,000 (DP 4-24), or in other embodiments between, for example 10 and 40. The miscibility properties of these materials with PVC are unknown. For those that are miscible, they are tested for efficacy as plasticizers, as indicated by the glass transition temperature and tensile strength of the resulting blends. A variety of 4-vinylphthalate monomers is investigated, by varying the alcohols making up the phthalate esters, and varying the molecular weight of these homopolymers. The TEMPO-based initiator is used for these polymerizations, as it is less costly than the H nitroxide-based initiators. It is understood that TEMPO, TIPNO, or many other nitroxide-based initiators can be used.

FIG. 9 shows Homopolymerization of 4-vinylphthalate esters by NMRP.

Random Co-Polymers

The bulky phthalic ester moieties may cause the homopolymers to be too rigid to exhibit satisfying plasticizing properties. Thus random copolymers prepared by a mixture of 4-vinylphthalate esters and styrenes or acrylates are prepared. Styrene as a co-monomer will statistically spread apart the bulky phthalate sidegroups, but the parent polymer backbone is essentially polystyrene.

FIG. 10 illustrates random copolymerization of 4-vinylphthalate esters and styrene by NMRP.

Alternatively, the use of acrylates will form copolymers that have polarity properties more closely related to the polyester plasticizers used today, without the susceptibility to backbone hydrolysis. It is possible that the acrylate “spacers” will act to isolate the bulky phthalate groups, allowing these copolymers to mimic traditional phthalate plasticizers when blended with PVC. As a rough guide in making the first selection of acrylates to be used, the optimal ratio determined by Lee of 5-7 methylene to ester units in polyesters to achieve maximal miscibility with PVC indicates that n-butyl acrylate is a good initial choice. A slightly branched alcohol such as isobutyl alcohol is another good choice. Again, a polymer with molecular weights of 2000-10000 (DP=4-24) is synthesized from 4-vinylphthalic esters and acrylates by NMRP. For these polymerizations using acrylates, our alpha-H nitroxide-based initiator is used, with a small amount of alpha-H nitroxide added to the polymerization mixture to ensure a controlled, living process. Note that copolymers with acrylates having a DP of 16-48 were generally used in the present study, but in practice the DP value could be from 2 to several thousand.

FIG. 11 shows random copolymerization of 4-vinylphthalate esters and acrylates by NMRP.

An alternative embodiment encompasses the use of 4-vinylphthalic anhydride as a co-monomer, to provide for the opportunity of post polymerization modification.

FIG. 12 shows random copolymerization of 4-vinylphthalate anhydride by NMRP: post-polymerization modification of the anhydride residues.

Variables: Variables include: alcohol on phthalate ester; molecular weight of polymer; identity and ratio of comonomer (for acrylates, alcohol of acrylate is another variable); ratio of polyphthalate plasticizer blended with PVC.

Solution cast films of PVC mixed with our designed phthalic ester polymers are prepared as described by Hakkarainen. These polymer blends are analyzed for miscibility and plasticizing effect, as well as stability.

Miscibility: Miscibility between the PVC and the polymeric phthalate plasticizers is determined by IR-spectroscopy and differential scanning calorimetry (DSC). Interactions between the CH₂—Cl— groups of PVC and the carbonyl groups of the polyesters are indicated by a shift of the carbonyl peak to lower wavenumbers. The existence of a single glass transition temperature is proof of full miscibility. Tensile Strength: The mechanical properties of PVC/poly(4-vinylphthalic ester) films is investigated by tensile strength analysis, a good method to determine elastomeric behavior. These new polymer blends is compared with PVC containing traditional phthalate plasticizers. Glass Transition Temperature: The plasticizing properties of the new polymer blends is probed by measuring the glass transition temperature, a good starting property is a T_{g} below −30° C. Stability and migration of polymeric plasticizers: Hydrolysis of PVC/poly(4-vinylphthalic ester) films is performed by aging the films for 10 weeks at 37° C. in water. The degradation product is analyzed by GC-MS. Mass loss and water absorption of the films will also be measured.

### BRIEF DESCRIPTION OF THE FIGURES

FIG. 1 illustrates a general scheme for the development of polymeric phthalate esters by NMRP (as plasticizers).

FIG. 2 shows Nitroxides and their corresponding N-alkoxyamine initiators for preparing polymeric phthalates.

FIG. 3 illustrates Stadler's synthesis of 4-vinylphthalic acid.

FIG. 4 illustrates a synthesis of 4-vinylphthalic esters 17 via Suzuki-Miyaura coupling.

FIG. 5 illustrates Denmark's use of vinylpolysiloxane 18 with tetra-butylammonium fluoride as an activator in a related palladium catalyzed coupling.

FIG. 6 shows Sonogashira route to vinyl phthalates.

FIG. 7 shows Diels-Alder approach to 4-vinylphthalates based on the work of Tsuge.

FIG. 8 illustrates routes to 4-vinylphthalic anhydride 26.

FIG. 9 shows Homopolymerization of 4-vinylphthalate esters by NMRP.

FIG. 10 illustrates random copolymerization of 4-vinylphthalate esters and styrene by NMRP.

FIG. 11 shows random copolymerization of 4-vinylphthalate esters and acrylates by NMRP.

FIG. 12 shows random copolymerization of 4-vinylphthalate anhydride by NMRP: post-polymerization modification of the anhydride residues.

FIG. 13 illustrates polymerization of a phthalate plasticizer mimic.

FIG. 14 illustrates preparation of a polymerizable phthalate plasticizer mimic.

FIG. 15 illustrates preparation of a triazole vinyl moiety.

FIG. 16 illustrates preparation of a triazole vinyl moiety.

FIG. 17 illustrates preparation of a triazole vinyl moiety.

FIG. 18 illustrates preparation of a triazole vinyl moiety.

FIG. 19 illustrates copolymerization of a triazole vinyl moiety with vinyl chloride.

FIG. 20 illustrates copolymerization of a triazole vinyl moiety with vinyl chloride.

FIG. 21 illustrates cycloaddition of azides and alkynes to prepare 1,2,3-triazoles covalently linked to PVC and bearing ortho esters containing branched alkoxy groups.

FIG. 22 illustrates thermal Huisgen cycloaddition to form triazole.

FIG. 23 illustrates thermal Huisgen 1,3-dipolar cycloaddition with a dialkyl acetylenedicarboxylate with diazide-terminated siloxane.

FIG. 24 illustrates thermal Huisgen 1,3-dipolar cycloaddition (in the absence of Cu) to form triazole.

FIG. 25 illustrates a synthesis of the monomethyl ester of 2-Butynedioic acid.

FIG. 26 illustrates a synthesis of a tosyl derivative of 2-Butynedioic acid (compound 17).

FIG. 27 illustrates a “click” cycloaddition of an alkyl azide to an ester of 2-Butynedioic acid (compound 16) to yield triazoles.

FIG. 28 illustrates a “click” cycloaddition of an alkyl azide to a tosyl derivative of 2-Butynedioic acid (compound 17) to yield triazoles.

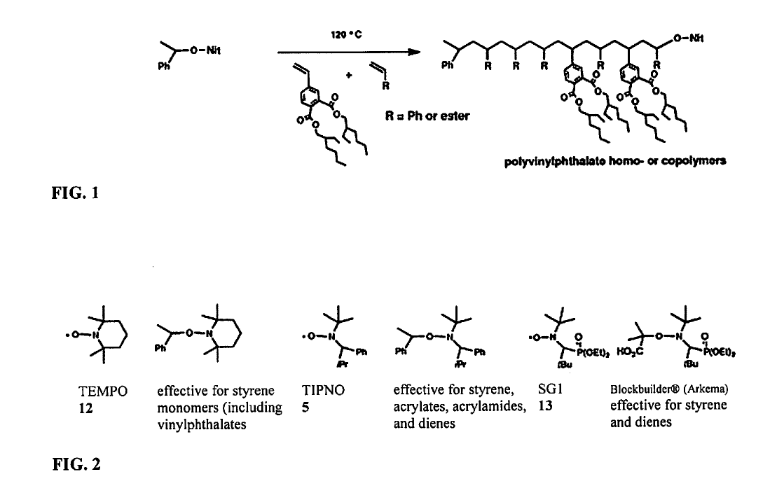

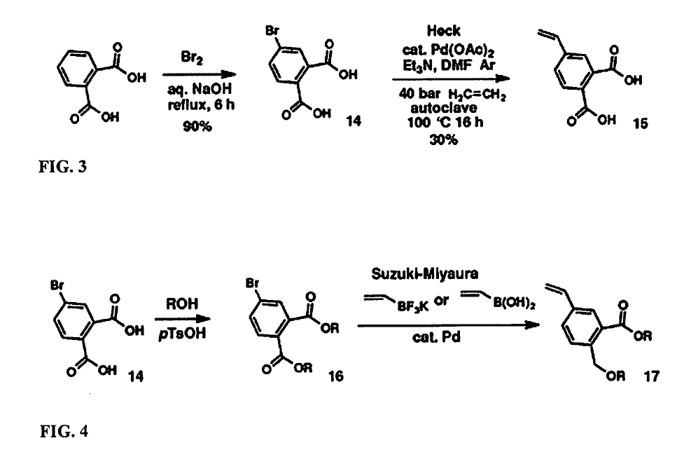

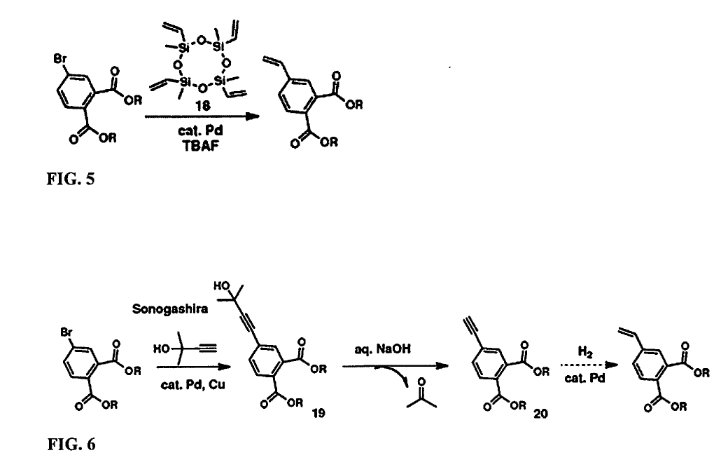

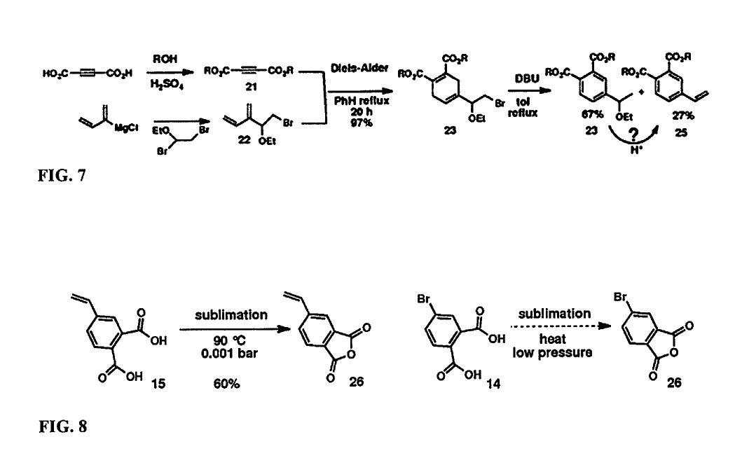

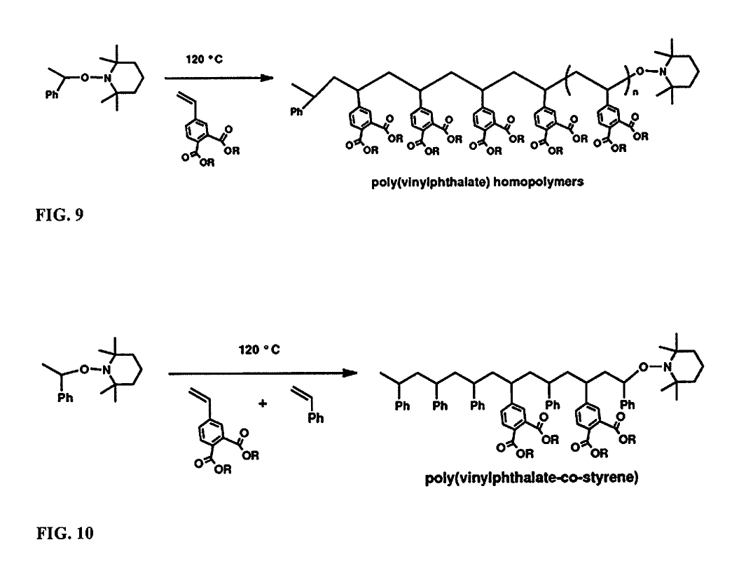

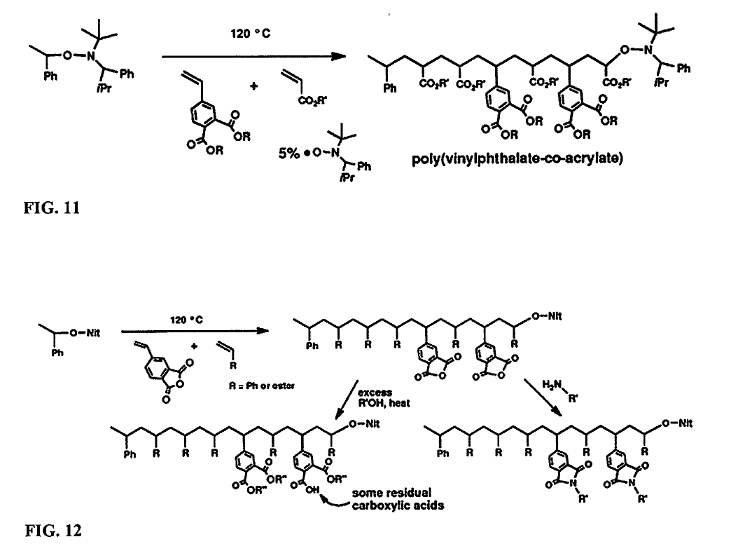

### GENERAL REPRESENTATIONS CONCERNING THE DISCLOSURE

All disclosures, publications and patent documents disclosed herein are hereby incorporated by reference to the fullest extent allowed by law. Other publications specifically incorporated by reference include: Navarro et al. ‘Phthalate Plasticizers Covalently Bound to PVC: Plasticization with Suppressed Migration.’ Macromolecules 2010, 43, 2377-2381; and Pawlak et al. Terrocene Bound Poly(vinyl chloride) as Ion to Electron Transducer in Electrochemical Ion Sensors.’ Analytical Chemistry 2010, 82 (16) 6887-6894; and Pawlak et al. ‘In situ surface functionalization of plasticized poly(vinyl chloride) membranes by ‘click chemistry’.’ Journal of Materials Chemistry 2012, 22 (25), 12796-12801; and Gonzaga et al. ‘Versatile, efficient derivatization of polysiloxanes via click technology.’ Chemical Communications 2009, (13) 1730-1732; and Grande et al. ‘Testing the functional tolerance of the Piers-Rubinsztajn reaction: a new strategy for functional silicones.’ Chemical Communications 2010, 46 (27), 4988-4990.

The embodiments disclosed in this specification are exemplary and do not limit the invention. Other embodiments can be utilized and changes can be made. As used in this specification, the singular forms “a”, “an”, and “the” include plural reference unless the context clearly dictates otherwise. Thus, for example, a reference to “a part” includes a plurality of such parts, and so forth. The term “comprises” and grammatical equivalents thereof are used in this specification to mean that, in addition to the features specifically identified, other features are optionally present. Where reference is made in this specification to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility). Where reference is made herein to “first” and “second” features, this is generally done for identification purposes; unless the context requires otherwise, the first and second features can be the same or different, and reference to a first feature does not mean that a second feature is necessarily present (though it may be present). Where reference is made herein to “a” or “an” feature, this includes the possibility that there are two or more such features. This specification incorporates by reference all documents referred to herein and all documents filed concurrently with this specification or filed previously in connection with this application, including but not limited to such documents which are open to public inspection with this specification.

### Definitions

The following words are used herein as follows:

The word Plasticizer is used herein to describe any substance added to a polymer to change brittleness, plasticity, viscosity, fluidity, hardness or alter another physical quality of the polymer.

The word Plastic refers to any polymeric organic amorphous solid compound that is moldable when heated and includes, for example acrylics, polyesters, silicones, polyurethanes, and halogenated plastics.

The word Hormone is used herein to describe any compound that interacts with the endocrine system of an animal.

The term Endocrine disruptor is used herein to describe any compound that interferes with the normal physiological functioning of the endocrine system of an animal.

To say that a plasticizer does not release phthalate esters, in this disclosure, means that it does not release an appreciable amount of phthalate esters, or alternatively that it releases less than the amount of phthalate esters that a commonly used traditional plasticizer will release over the same period of time; for example no more than 10% or 20%. In other embodiments it may release no more than 30%, 40%, 50%, 60%, 70% or no more than 80% of phthalate esters that a commonly used traditional plasticizer will release over the same period of time. For example a plasticizer made of short polymers consisting of a covalent carbon chain backbone bearing phthalate ester side-groups may release less than 30% of the phthalate esters that would be released by a plasticizer not made of short polymers consisting of a covalent carbon chain backbone bearing phthalate ester side-groups.

A “click” reaction is a Cu-assisted azide-alkyne cycloaddition.

### DETAILED DESCRIPTION OF THE INVENTION

The invention encompasses a novel, simple and economical method of covalently attaching a phthalate ester mimic to polymers such as PVC, allowing plasticization of PVC and other polymers to produce commercial polymers from which endocrine disruption chemicals do not leach (or leach in very small quantities) from the polymer matrix. The invention also encompasses the products of such reactions and methods for making and using such compounds and plastics (such as PVC) blended with such compounds. Plastics and polymers that may be plasticized by the method of the invention include, for example, polyvinyl chloride, polyvinyl acetate, rubbers, cellulose plastics, and polyurethane.

In the method of the invention, an azide-alkyne Huisgen cycloaddition using dialkyl acetylenedicarboxylates allows cycloaddition to occur under very mild thermal conditions, such as room temperature, such as between 10° C. and 20° C., for example below 40° C., below 30° C., below 20° C., below 15° C., or below 10° C. In certain embodiments the method is carried out in the absence of a catalyst, for example in the absence of a metal catalyst, for example in the absence of a copper catalyst.

A novel, simple and economical route to covalently attach a phthalate ester mimic to PVC is described, allowing plasticization of PVC without the danger of Endocrine Disruption Chemicals leaching from the polymer matrix. An azide-alkyne Husigen cycloaddition (in the absence of a metal catalyst, e.g., a copper catalyst) using dialkyl acetylenedicarboxylates allows cycloaddition to occur under very mild thermal conditions.

In most embodiments, the azide-alkyne Huisgen cycloaddition is Cu free and performed at low temperatures, e.g., below 20° C. or 10° C., but I other embodiments the reaction is carried out using a catalyst, such as using a metal catalyst such as Cu, and may (separately or in addition) be carried out at higher temperatures, for example between 20° C. and 60° C., for example above 10° C., above 20° C., above 30° C., above 40° C., or above 50° C.

The method of the invention may be performed by the chemical modification of already formed polymers such as polyvinyl chloride, or in other embodiments, may be performed by the modification of monomers prior to polymerization by the cycloaddition of dialkyl acetylenedicarboxylates.

In most embodiments, allylic sites on a polymer or monomer (to be polymerized) may be targets for azide displacement. Allylic C—H bonds are about 15% weaker than the normal C—H bonds and the most labile electrophilic chloride sites on PVC are secondary allylic chlorides. However, in other embodiments, particularly with PVC or other polymers that do not have many allylic sites, regular alkyl secondary chlorides can be displaced by azide as well as allylic chlorides.

An important embodiment of the invention is the discovery of a method for the production of covalently-bonded mimics of phthalate plasticizers, the method comprising performing an azide-alkyne Huisgen cycloaddition reaction of dialkyl acetylenedicarboxylates with azide-functionalized PVC.

In some embodiments the method of thermal azide-alkyne Huisgen cycloaddition may be performed in the absence of a copper catalyst. The methods may be performed without any external catalyst, for example without a metal catalyst, for example without a copper catalyst. In some embodiments the method of thermal azide-alkyne Huisgen cycloaddition may be performed under very mild thermal conditions.

In some embodiments the method may be performed wherein the thermal conditions are ambient conditions (room temperature) and the time of reaction is extended. For example, the thermal conditions of the reaction may be between 5° C. and 35° C., between 10° C. and 30° C., between 15° C. and 25° C. In other embodiments the thermal conditions of the reaction may be between 10° C. and 100° C., 30° C. and 75° C., 25° C. and 60° C., 10° C. and 20° C., or simply at room temperature. Alternatively the temperature at which the reaction is performed bay be below 40° C., below 30° C., below 20° C., below 15° C., or below 10° C.

In some embodiments the reaction requires at least 4 hours to proceed to at least 80% completion. In others it requires at least 6 hours to proceed to at least 95% completion. In other embodiments, to reach 90% completion, the reaction may require, 2, 4, 6, 8, 10, 12, 14, 16, 18, 20, 22, or 24 hours or may simply be performed overnight.

The invention encompasses polymers (e.g., for example, polyvinyl chloride, polyvinyl acetate, rubbers, cellulose plastics, and polyurethane) to which a phthalate ester mimic is covalently attached, allowing plasticization of polymers such as PVC without the danger of Endocrine Disruption Chemicals leaching from the polymer matrix.

The invention includes products-by-process comprising a polymer plasticized by the method of the invention.

An azide-alkyne Huisgen cycloaddition in the absence of a metal (e.g., copper) catalyst using dialkyl acetylenedicarboxylates allows this cycloaddition to occur under very mild thermal conditions.

Azide-alkyne Huisgen 1,3-dipolar cycloaddition reactions utilizing very electron deficient acetylenes with alkyl azides can take place at room temperature in the absence of a metal catalyst. Electron-poor alkynes bearing esters, carboxylic acids, amides and sulfones used in Cu-free “click” cycloadditions at ambient temperature are suitable for widespread use in organic synthesis, biomolecular investigations and the development of new materials.

A particular example focuses on polyvinyl chloride. The problem of leaching of endocrine disrupters from plasticized compounds is solved by the formation of covalently-bound 1,2,3-Triazole Phthalate Mimics. Treatment with sodium azide produces PVC in which some of the chloride has been replaced with azide. Reaction with dialkyl acetylenedicarboxylates will give 1,2,3-triazoles bearing ortho esters. Esters made of branched alcohols form mimics of phthalate ester plasticizers, covalently linked to PVC. Migration of these plasticizer mimics is completely suppressed; hydrolysis will release only alcohols rather than phthalates. These triazoles bearing branched esters prove to be effective plasticizers and this approach may be used to replace the use of millions of tons of phthalate esters produced every year as plasticizers.

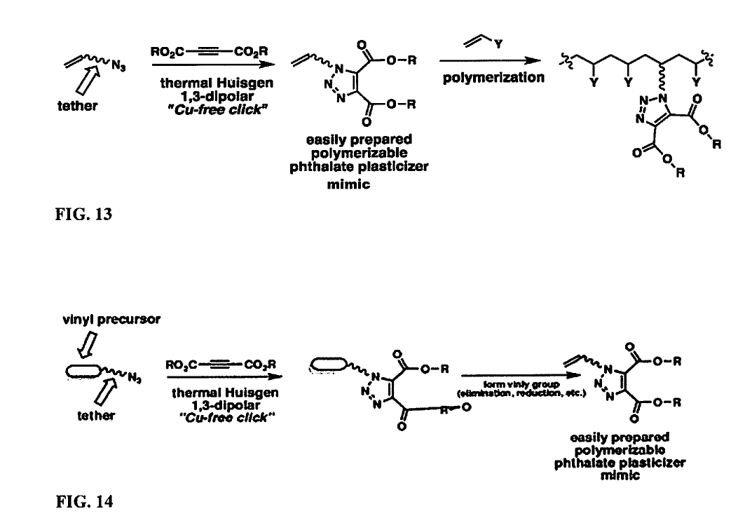

Additional embodiments of the invention include further monomers beyond vinyl bearing 1,2,3-Triazole Phthalate Mimics prepared by Azide/Alkyne Cycloaddition. Whereas the original invention encompassed a vinyl monomer that can be modified to bear a phthalate mimic consisting of a triazole (see FIG. 13).

Alternative embodiments may expand this to the use of vinyl precursors, that undergo the dipolar cycloaddition prior to formation of the vinyl group (see FIG. 14).

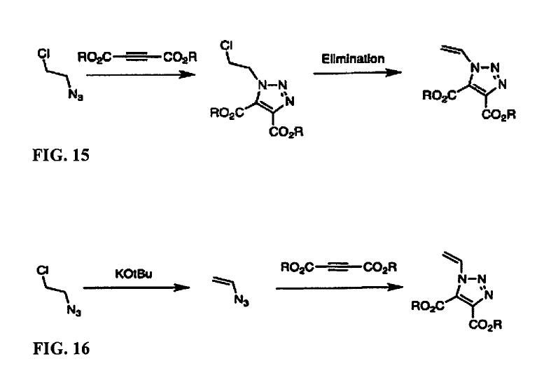

An example of this is following sequence: triazole formation followed by elimination (upon treatment with base, heat, or other stimulus) to form the vinyl moiety see FIG. 15.

Vinyl azide is produced by a similar reaction, and provides an alternative route (see FIG. 16).

In the basic embodiment, vinyl acetate analogues are used as a typical monomer class. In other embodiments, this is expanded to include vinyl ethers, to provide electron rich monomers that arc easily copolymerized with vinyl chloride:

TABLE 2

| vinyl acetate analogues | vinyl ethers |
|---|---|
| 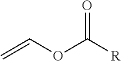 | 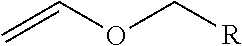 |
| 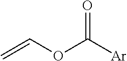 | 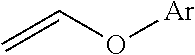 |

R = alkyl bearing N₃
Ar = aryl bearing N₃

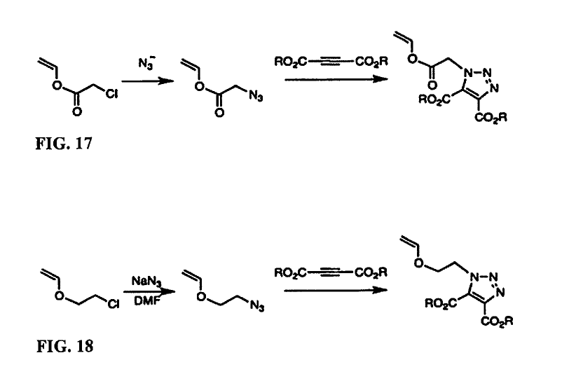

For example, the widely available vinyl chloroacetate can be converted to the azide and then the triazole (see FIG. 17).

and the commodity chemical 2-chloroethyl vinyl ether can be converted into the azide and then the triazole (see FIG. 18).

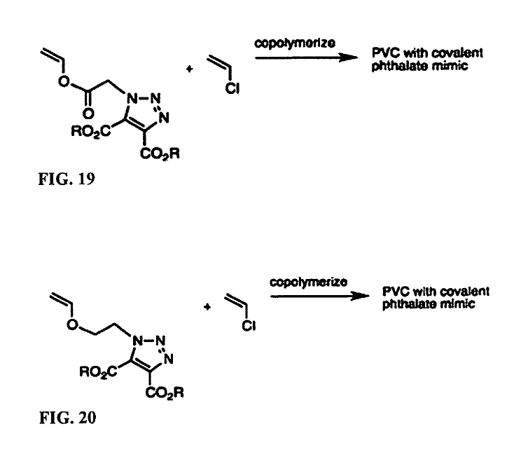

These electron-rich alkenes can undergo copolymerization in an uncontrolled fashion (as a bulk solution, dispersion, inversion dispersion, emulsion, etc.) or in a controlled polymerization with vinyl acetate to give PVC with covalently attached phthalate mimics (see FIG. 19).

Likewise, the copolymerization of vinyl chloride with vinyl ethers will also generate covalently attached phthalate mimics (see FIG. 20).

Another embodiment encompasses the use of olefin monomers. A thermal azide-alkyne Huisgen cycloaddition (preferably in the absences of copper catalyst) using dialkyl acetylenedicarboxylates allows cycloaddition to be carried out on olefin monomers bearing azides under very mild thermal conditions. Olefin monomers may be, for example, acrylates, acrylamides, methacrylates, styrenes, vinyl acetate (and derivatives, such as alpha-chlorovinyl acetate), vinyl chloride, dienes, acrylonitrile, maleimides, norbornenes, vinyl ethers, fumarates, vinyl ketones, 1-alkenes, or maleic anhydrides.

In another alternative embodiment of the Cu-free “click” cycloadditions, the order of “click” reaction may be changed, and rather than having olefin monomers functionalized by azide, and then clicked, an alternative method is to functionalize with azide, click, and then form the olefin group.

In various embodiments vinyl chlorides may be used as monomers, but other monomers may be used, for example vinyl ethers and vinyl acetates. This is a useful embodiment since copolymerization of the electron-rich olefin monomers with vinyl chloride should be particularly effective.

A further embodiment provides monomers bearing 1,2,3-triazole phthalate mimics prepared by azide/alkyne cycloaddition. The method encompasses formation of monomers bearing phthalate ester mimics, which can be used in a variety of polymerization reactions to incorporate covalently bonded plasticizers into polymer chains. A simple thermal reaction at or near room temperature in the absence of catalyst is used to prepare the polymerizable monomers from readily available starting materials. A variety of olefin monomers are envisioned. A Huisgen 1,3-dipolar cycloaddition of azide and alkynes is utilized to prepare 1,2,3-triazoles bearing ortho esters containing branched alkoxy groups, to create mimics of phthalate esters into monomers, which upon polymerization will result in plasticizer mimics covalently incorporated into a variety of polymers. hydrolysis will release only alcohols rather than phthalates. The azide group is easily introduced into molecules by SN2 reaction. The azide group cannot be carried through free radical polymerization, as carbon radicals add to azides. However, 1,3-dipolar cycloaddition with dialkyl acetylenedicarboxylates will provide aromatic triazole products, which will be completely compatible with free radical polymerization reactions. To date, several azide-containing polymers have been utilized: Cu catalyzed “click” cycloaddition is carried out prior (or concurrently) to their use as monomers. The styrene derivative benzyl azide18 (3) has been utilized in ATRP radical polymerizations, with concurrent Cu-catalyzed “click” cycloaddition. Multiple references19 to methacrylates (4) have been reported, to make triazole-containing comonomers, which are then used in ATRP or RAFT radical polymerizations. In one case, the azide monomer (4) (n=2) was successfully utilized in both ATRP and RAFT polymerizations at 60° C. and 65° C., to form azide-functionalized polymers, followed by reactions of the pendant azides to prepare specialized surface coatings. Methacrylate (5) bearing an aryl azide ester (21) has been utilized in Cu catalyzed click chemistry followed by RAFT polymerization. Methacryl amide has been utilized in Cu-catalyzed click reactions followed by both ATRP22 and RAFT23 polymerizations. Azide-containing monomers will be converted under mild, Cu-free conditions to the corresponding triazoles, for subsequent use in random copolymerizations. For example, benzyl azide 3 will be converted to the triazole styrene 7, which can be used to covalently incorporate covalently bonded plasticizers as random copolymers. In a second example, acrylate or methacrylates are converted to triazoles (8) for subsequent use as monomeric polymerizable plasticizers. Another easily accessed acrylate or methacrylate is the azide 9 obtained by azide opening of the epoxide24 of glycidyl acrylate or glycidyl methacrylate. This general approach can be envisioned to prepare monomeric derivatives of styrenes, acrylates and methacrylates, acrylamides and methacrylamides, maleimides and even vinyl acetate analogues, as shown in Table 3.

TABLE 3
Common Olefin Monomer Classes Amenable to Triazole Attachment

| styrenes | acrylates, methacrylates | acrylamides, methacrylamides | | malemides | vinyl acetate analogues |
|---|---|---|---|---|---|
| 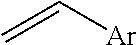 | 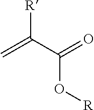 | 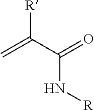 | 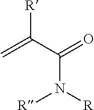 | 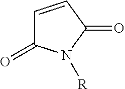 | 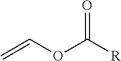 |
| | 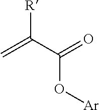 | 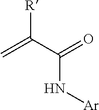 | 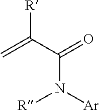 | 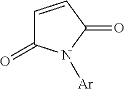 | 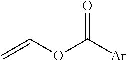 |

R = alkyl bearing N₃
Ar = aryl bearing N₃

In order to mimic a number of different phthalate ester plasticizers, the alcohol on the ester moieties of the triazole can be varied as shown in Table 4.

TABLE 4

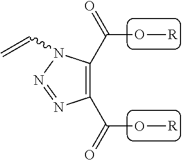

| | Phthalate |
|---|---|
| | being |
| Alcohol | mimicked |
| 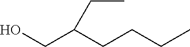 | DEHP |
| 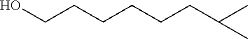 | DINP |
| 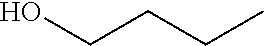 | DBP |
| 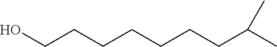 | DIDP |
| 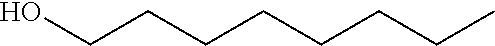 | DOP |
| 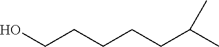 | DIOP |
| 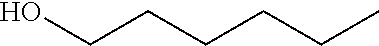 | DNHP |
| 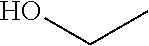 | DEP |
| 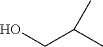 | DIBP |
| 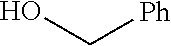 | (half of) BBzP |

Materials and Methods

The present method employing a mild “click” approach to phthalate ester mimics is a simple, economical and scalable alternative to the use of phthalate plasticizers, while mitigating the health hazards associated with the use of phthalates.

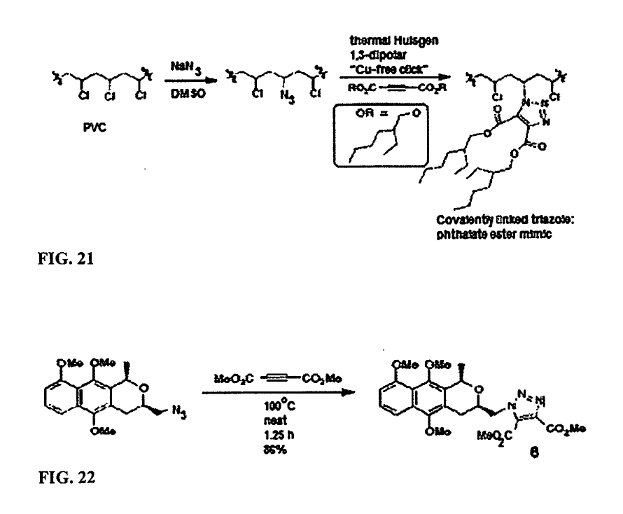

The powerful Huisgen 1,3-dipolar cycloaddition of azide and alkynes is utilized to prepare 1,2,3-triazoles bearing ortho esters containing branched alkoxy groups, to prepare mimics of phthalate esters covalently linked to PVC (see FIG. 21). Thus migration is completely suppressed; hydrolysis will release only alcohols rather than phthalates. Thus degradation products pose no danger of being metabolized to form Endocrine Disruptor Compounds.

Azide is a fairly good nucleophile. The polarity of the solvent, temperature, reaction time and stoichiometry of azide utilized is critical in controlling the amount of SN2 substitution reaction compared to elimination. DMF is usually the solvent of choice, however use of the less polar solvent cyclohexanone results in a slower reaction, allowing stereoselective displacement to occur at the mm triad of mmmr tetrads, and the rm diad of rrmr pentads. Surface modification by azide displacement of chloride has also been studied on PVC films.

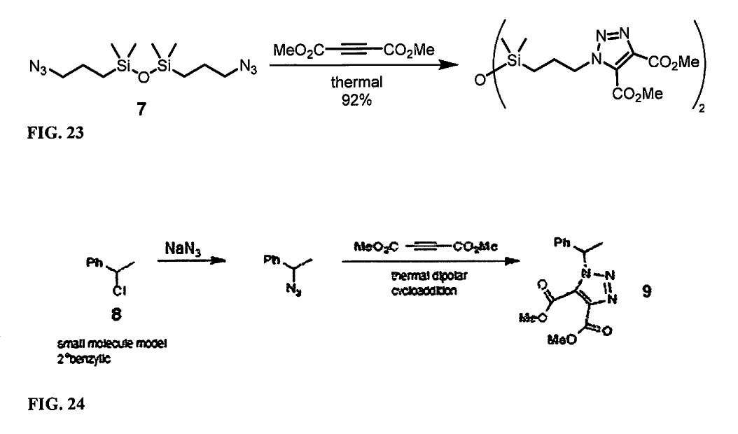

Cu-assisted azide-alkyne cycloaddition (commonly known as a “click” reaction) has become an extremely popular method to reliably form triazoles from organoazides and terminal alkynes. Bakker has utilized Cu-catalyzed “click” chemistry to surface functionalize PVC bearing azide groups with ferrocene and fluorescent dyes using terminal alkynes, with the goal of tuning the electronic properties of the membrane solution interface of ion sensors. However, the use of a copper catalyst, even in trace amounts, is not desirable for a commodity product with applications in the construction of medical devices and food and drink packaging. Copper free variations utilizing cyclooctynes have enjoyed popularity in both biology and materials science, but is restricted to the use of very specialized 8-memberred ring cyclic alkynes. By utilizing alkynes substituted on both ends by an ester, the alkyne partner becomes extremely electrophilic, lowering the LUMO, and thus enhancing the 1,3-dipolar cycloaddition. For example, Brimble utilized dimethyl acetylenedicarboxylate to carry out thermal Huisgen cycloaddition in neat excess alkyne at 100° C. to form triazole (6) (see FIG. 22). In a second example, Brook has carried out thermal Huisgen 1,3-dipolar cycloaddition with dialkyl acetylenedicarboxylates with diazide-terminated siloxanes such as (7) (see FIG. 23). Another advantage of utilizing dialkyl acetylenedicarboxylates is that the alkyne is symmetrical, thus avoiding mixtures of regioisomers often observed in thermal Huisgen cycloadditions.

Results

Given that dehydrochlorination of HCl from PVC occurs thermally by multiple mechanisms, azide substitution in polar solvents is likely to occur at allylic chlorides by an S_{N}2′ mechanism prior to S_{N}2 at secondary alkyl chlorides. Thus the most labile electrophilic chloride sites on PVC are secondary allylic chlorides.

As a small molecule model, the inventors utilized the secondary benzylic chloride 1-chloro-1-phenylethane (8): azide displacement of chloride using NaN3 on Amberlite resin was straightforward.

The researchers then carried out the key thermal Huisgen 1,3-dipolar cycloaddition (in the absence of Cu) to form triazole (9) (see FIG. 24); the results are summarized in Table 5. The reaction was monitored by both TLC and 1H-NMR. Following the general procedure of Brimble, the researchers started out with a large excess of the electron poor dimethyl acetylenedicarboxylate at 100° C.: the reaction went to completion in under an hour. The researchers then reduced the number of equivalents as well as the temperature.

The researchers were excited to find that the reaction goes to completion with only a slight excess of alkyne, and the temperature can be reduced to ambient conditions (room temperature), albeit requiring an overnight reaction time. The reaction proceeds equally well neat, or with dcutcrochloroform as the solvent.

As a second model, the researchers also converted geranyl chloride (a primary allylic chloride) to the azide. Cu-free “click” reaction with dimethyl acetylenedicarboxylate gave complete conversion to the triazole at room temperature overnight, isolated in 83% yield,

TABLE 5

| Equivalents of dimethyl | | | |
|---|---|---|---|
| acethylenedicarboxylate | Solvent | Temperature | Time |
| 5 [symbol] | neat | 100° C. | 40 min |
| 5 | neat | 50° C. | 40 min |
| 5 | neat | RT | 40 min |
| 1.5 | neat | RT | overnight |
| 1.5 | CDCl₃ | RT | overnight |

The next step is performing an azidization of PVC: this reaction is usually monitored by IR. Bakker has determined reaction times for azide displacement of chloride in commercial PVC (purchased from Sigma-Aldrich) to obtain 2-6% azidification. In addition, 1H-NMR and elemental analysis will provide additional tools to determine conversion.

The key thermal cycloaddition between dialkyl acetylenedicarboxylates is carried out in solution, followed by precipitation of the polymer (typically PVC is dissolved in THF, and precipitated by addition of methanol, however use of 1,2-dichlorobenzene as solvent followed by addition of toluene has also been used).

The researchers chose dimethyl acetylenedicarboxylate for our initial experiments, to generate simple NMR spectra. The cycloaddition described may be extended to PVCazide, branched alkyl esters related to the most common phthalate esters (see Table 6).

TABLE 6

| | Phthalate |
|---|---|
| | being |
| Alcohol | mimicked |
| 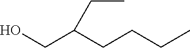 | DEHP |
| 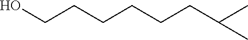 | DINP |
| 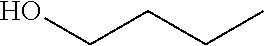 | DBP |
| 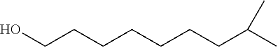 | DIDP |
| 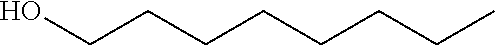 | DOP |
| 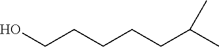 | DIOP |
| 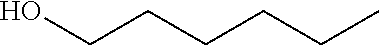 | DNHP |
| 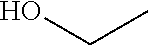 | DEP |
| 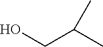 | DIBP |
| 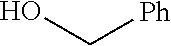 | (half of) BBzP |

Characterization of the Modified PVC Polymers and their Plasticizing Properties

The characterization of the polymers with covalently linked triazoles uses IR, 1H NMR spectroscopy and GPC (size exclusion chromatography) for determination of percent conversion, molecular weight, and polydispersity. Modified polymers are analyzed for miscibility and homogeneity over time, as well as chemical stability and resistance to migration as follows:

Miscibility (measured by IR) of the derivatized PVC with untreated PVC may be determined by IR spectroscopy.

Miscibility (measured by DSC): the existence of a single glass transition temperature determined by differential scanning calorimetry (DSC) for a polymer blend is the least ambiguous evidence for miscibility. For the most promising samples, additional information regarding miscibility and morphology may be be obtained using scanning electron microscopy (SEM).

Plasticization as measured by depressed glass transition temperatures: the plasticizing properties of the new polymer blends may be be probed by measuring the glass transition (Tg) temperature, the depression of which is a reliable quantitative measure of the increased flexibility, or softening of the polymer blend.

Stability and migration resistance of covalent plasticizer mimics and their possible degradation products: Hydrolysis of modified PVC films may be performed by aging the films for 10 weeks at 37° C., and at 70° C. in water at neutral and low pH following ASTM methods for extractability in hexanes and methanol. The degradation products can be analyzed by GC-MS. Mass loss and water absorption of the films can also be measured.

Long-term homogeneity of the PVC/polymeric plasticizer blends: the stability of the modified PVC materials is studied as a function of time, to determine if phase separation occurs with aging.

Further Applications of the Present Invention.

The disclosed methods may be employed for applications well beyond phthalate mimics, and the thermal 1,3-dipolar Huisgen azide-alkyne cycloaddition at ambient temperature in the absence of copper has many important applications that are enabled using the disclosed methods. The surprisingly mild conditions required to effect thermal “click” cycloaddition of alkyl azides and very electron-poor alkynes in the absence of a copper catalyst has been overlooked by the community of synthetic chemists, bioorganic chemists and materials chemists.

From our work it is apparent that a single electron-withdrawing group is sometimes sufficient to effect “thermal” Huisgen cycloaddition at room temperature, but often these reactions require extended reaction times, or give low yields. Thus development of electron-poor alkynes bearing two electron-withdrawing groups ensures easy cycloaddition at ambient temperatures in reliably high yields. Versatility in attaching functionalizable handles allows these alkynes to be utilized for Cu-free “click” reactions for a variety of applications. For this purpose, two highly electron deficient alkynes: ester, acid substituted alkyne 16, and sulfone, acid-substituted alkyne 17 are proposed as general starting points for ambient temperature “thermal” click reactions with alkyl azides.

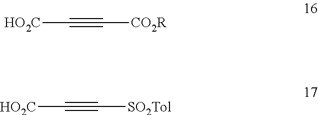

The carboxylic acid can be converted to an amide or ester to allow conjugation of biomolecules, or more generally to alcohol or amine functional groups for a multitude of applications.

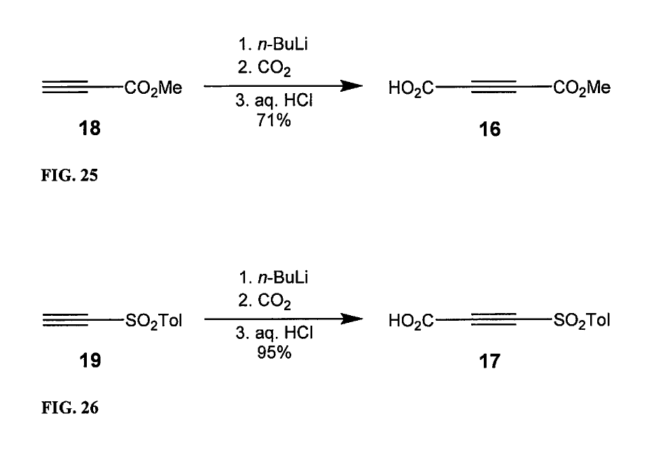

The synthesis of each alkyne is straightforward: Hall has described the synthesis of the methyl ester of 16 starting from commercially available methyl propynoate 18 in 71% yield (see FIG. 25). Likewise, Corey described the synthesis of sulfone 17 from p-toluenesulfonylacetylene 19 in his 1988 synthesis of forskolin (see FIG. 26).

With these two very electron deficient alkynes, room temperature “click” reactions without copper catalyst can be tested, both as the free carboxylic acids, and as conjugates with a variety of small organic molecules. Reactions in water as well as organic solvents are being investigated. The alkyl group of the ester in alkyne 16 can be manipulated to tune the solubility in water or organic solvents. Using the present disclosure, these methods can be extended to biologically interesting molecules, such as glycopeptides and biomaterial hybrids.

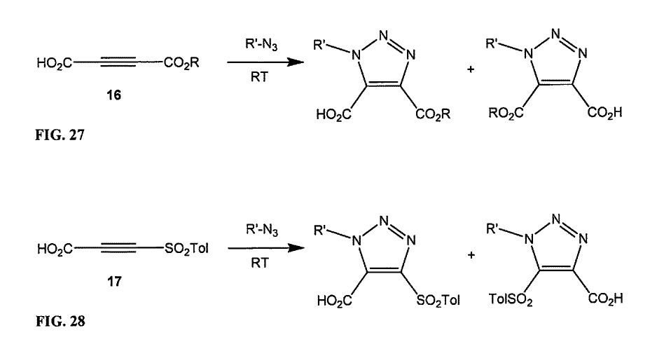

Cycloaddition with alkyl azides provides the expected triazoles at room temperature (see FIGS. 27 and 28). The regioselectivity may be determined for small molecules: this regiochemistry may or may not be important for larger molecular assemblies. The thermal stability of these triazoles is high.

Using the methods of the invention, it is believed that these highly electron deficient alkynes will add to the tool-box of readily available reagents for coupling azides to alkynes under copper-free conditions at room temperature.

In summary, the ‘thermal’ azide-alkyne Huisgen cycloaddition reaction of dialkyl acetylenedicarboxylates with azide-functionalized PVC is carried out to prepare covalently-bonded mimics of phthalate plasticizers to provide effective plasticizers.

This methodology could replace the millions of tons of phthalate esters produced every year. As phthalate esters migrate out of PVC during both the consumer lifetime of commercial products, and for years afterwards as the PVC undergoes degradation, massive amounts of phthalates are introduced into the environment, and become metabolized to form Endocrine Disrupting Chemicals when ingested or absorbed by mammals.

This “click” approach to phthalate mimics provides a simple, economical and scalable alternative.

Equally as important, the development of two versatile electron poor alkynes 16 and 17 for the general application of Cu-free “click” Huisgen cycloaddition at ambient temperature is may be use in organic synthesis, biomolecular investigations and the development of new materials.

1.-20. (canceled)

21. A dialkylcarboxylate-aromatic-functionalized halohydrocarbon or hydrocarbon polymer.

22. A dialkylcarboxylate-1,2,3-triazole-functionalized halohydrocarbon polymer of claim 21.

23. A 4,5-dialkylcarboxylate-1,2,3-triazole-functionalized chlorohydrocarbon polymer of claim 22.

24. A 4,5-dialkylcarboxylate-1,2,3-triazole-functionalized polyvinyl chloride of claim 22.

25. The 4,5-dialkylcarboxylate-1,2,3-triazole-functionalized polyvinyl chloride of claim 24,
wherein the 1-nitrogen of the triazole is covalently bonded to the polyvinyl chloride carbon backbone.

26. The polymer of claim 22 comprising the structure 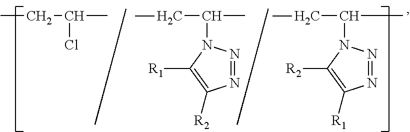
wherein R₁ and R₂ are independently selected from the group consisting of R₃O(C═O)—, R₄O(C═O)—, and tosyl (Ts),
wherein R₃ is selected from the group consisting of hydrogen (H), alkyl, branched alkyl, phenyl, benzyl, and cycloaliphatic, and
wherein R₄ is selected from the group consisting of alkyl, branched alkyl, benzyl, and cycloaliphatic.

27. The polymer of claim 26,
wherein R₃ is selected from the group consisting of hydrogen (H), alkyl, branched alkyl, phenyl, and benzyl and
wherein R₄ is selected from the group consisting of alkyl, branched alkyl, and benzyl.

28. The polymer of claim 26,
wherein R₃ is selected from the group consisting of alkyl and branched alkyl and
wherein R₄ is selected from the group consisting of alkyl and branched alkyl.

29. The polymer of claim 26,
wherein R₃ is selected from the group consisting of alkyl of from 4 to 15 carbons and branched alkyl of from 4 to 15 carbons and
wherein R₄ is selected from the group consisting of alkyl of from 4 to 15 carbons and branched alkyl of from 4 to 15 carbons.

30. The polymer of claim 26,
wherein R₃ is selected from the group consisting of methyl, ethyl, 1-methylethyl (isopropyl), n-butyl, 2-methylpropyl (isobutyl), n-hexyl, n-octyl, 2-ethylhexyl, 6-methylheptyl (isooctyl), 7-methyloctyl (isononyl), 3,3,5-trimethylhexyl, 8-methylnonyl (isodecyl), and benzyl and
wherein R₄ is selected from the group consisting of methyl, ethyl, 1-methylethyl (isopropyl), n-butyl, 2-methylpropyl (isobutyl), n-hexyl, n-octyl, 2-ethylhexyl, 6-methylheptyl (isooctyl), 7-methyloctyl (isononyl), 3,3,5-trimethylhexyl, 8-methylnonyl (isodecyl), and benzyl.

31. The polymer of claim 26,
wherein R₃ is selected from the group consisting of methyl, ethyl, n-butyl, 2-methylpropyl (isobutyl), n-hexyl, n-octyl, 2-ethylhexyl, 6-methylheptyl (isooctyl), 7-methyloctyl (isononyl), 8-methylnonyl (isodecyl), and benzyl and
wherein R₄ is selected from the group consisting of methyl, ethyl, n-butyl, 2-methylpropyl (isobutyl), n-hexyl, n-octyl, 2-ethylhexyl, 6-methylheptyl (isooctyl), 7-methyloctyl (isononyl), 8-methylnonyl (isodecyl), and benzyl.

32. The polymer of claim 27,
wherein R₁ and R₂ are different.

33. The polymer of claim 27,
wherein R₁ and R₂ are the same.

34. The polymer of claim 27,
wherein R₁ is R₃O(C═O)—,
wherein R₂ is R₄O(C═O)—, and
wherein R₃ and R₄ are different.

35. The polymer of claim 27,
wherein R₁ is HO(C═O)— and
wherein R₂ is R₃O(C═O)—.

36. The polymer of claim 27,
wherein R₃ is benzyl and
wherein R₄ is n-butyl.

37. The polymer of claim 27,
wherein R₁ is R₄O(C═O)— and
wherein R₂ is R₄O(C═O)—.

38. The polymer of claim 27,
wherein R₁ is H₃CO(C═O)— and
wherein R₂ is H₃CO(C═O)—.

39. The polymer of claim 27,
wherein R₃ is ethyl and
wherein R₄ is ethyl.

40. The polymer of claim 27,
wherein R₃ is 1-methylethyl (isopropyl) and
wherein R₄ is 1-methylethyl (isopropyl).

41. The polymer of claim 27,
wherein R₃ is 2-methylpropyl (isobutyl) and
wherein R₄ is 2-methylpropyl (isobutyl).

42. The polymer of claim 27,
wherein R₃ is 2-ethylhexyl and
wherein R₄ is 2-ethylhexyl.

43. The polymer of claim 27,
wherein R₃ is 3,3,5-trimethylhexyl and
wherein R₄ is 3,3,5-trimethylhexyl.

44. The polymer of claim 27,
wherein R₃ is 7-methyloctyl (isononyl) and
wherein R₄ is 7-methyloctyl (isononyl).

45. The polymer of claim 27,
wherein R₃ is 8-methylnonyl (isodecyl) and
wherein R₄ is 8-methylnonyl (isodecyl).

46. The polymer of claim 27,
wherein R₃ is branched alkyl of 10 carbons and
wherein R₄ is branched alkyl of 10 carbons.

47. The polymer of claim 27,
wherein R₁ is HO(C═O)— and
wherein R₂ is tosyl.

48. The polymer of claim 27,
wherein R₁ is tosyl and
wherein R₂ is tosyl.

49.-69. (canceled)
